(19) United States
(12) Patent Application Publication
(10) Pub. No.: US 2020/0379636 A1
(43) Pub. Date: Dec. 3, 2020

(54) IMAGE PROCESSING APPARATUS, IMAGE PROCESSING METHOD, AND NON-TRANSITORY COMPUTER-READABLE STORAGE MEDIUM

(71) Applicant: CANON KABUSHIKI KAISHA, Tokyo (JP)

(72) Inventor: Toru Takasawa, Kawasaki-shi (JP)

(21) Appl. No.: 16/884,221

(22) Filed: May 27, 2020

(30) Foreign Application Priority Data
Jun. 3, 2019 (JP) 2019-103946

(51) Int. Cl. G06F 3/0484; G11B 27/34; G06F 3/0488; G06F 3/0483; G16H 30/40

(52) U.S. Cl. CPC G06F 3/04847; G11B 27/34; G16H 30/40; G06F 3/0483; G06F 3/0488

(57) ABSTRACT

An image processing apparatus comprising: an extraction unit configured to extract at least one region of interest with respect to each frame image of a series of frame images of an object; a calculation unit configured to calculate a feature amount of the region of interest; and a display control unit configured to cause a display unit to display the feature amount as a time series graph, wherein when at least some of the series of frame images are to be played back as a moving image, the display control unit causes an index which represents a position corresponding to the displayed frame image to be displayed on the graph.

### BACKGROUND OF THE INVENTION

### Field of the Invention

The present invention relates to an image processing apparatus, an image processing method, and a non-transitory computer-readable storage medium, and more particularly to a technique that allows a required range to be easily selected from a series of frames obtained by moving image capturing.

### Description of the Related Art

Conventionally, a radiation imaging system using a radiation imaging apparatus that irradiates an object with radiation from a radiation generation apparatus, digitizes a radiation image which shows an intensity distribution of the radiation transmitted through the object, performs image processing on the digitized radiation image, and generates a clear radiation image has been commercialized.

An FPD (Flat Panel Detector) in which solid-state image sensors are two-dimensionally arrayed in a matrix is known as a radiation detector. An FPD can perform not only still image capturing, but also moving image capturing of the movement of an object by performing a plurality of imaging operations in a period of 1 sec by using the quick responsiveness of image data read/erase operations to continuously emit radiation pulses from a radiation source in accordance with the read/erase timings of the FPD. A series of a plurality of frame images obtained from the imaging can be sequentially displayed on a monitor of an inspection apparatus so that a radiographer can recognize the series of movements of the object. After the completion of the inspection, the series of the plurality of frame images can be transferred to a medical image management system called PACS (Picture Archiving and Communication Systems) via an in-hospital network so that the series of frame images obtained from the moving image capturing can be analyzed on PACS to generate diagnosis support information, and the generated diagnosis support information can be provided to a doctor for early diagnosis.

In this case, since a moving image is formed by a plurality of frame images, the information amount generated for each imaging operation will increase, and the amount of information the doctor will use on PACS to make a diagnosis will also increase. In contrast, Japanese Patent Laid-Open No. 2018-110637 discloses that the information amount of a radiation imaging system is reduced by extracting some of the frame images of interest (for example, frame images corresponding to a single respiratory cycle) from the series of frame images of an object and transferring only some of the frame images of interest of a range that has been extracted.

However, it is difficult to discriminate the state of the object captured in the displayed frame image at the time of playback of the moving image in the technique disclosed in Japanese Patent Laid-Open No. 2018-110637. That is, it is problematically difficult to easily grasp the correspondence relationship between the state of the object and each frame image to be played back in the moving image.

In consideration of the above problem, the present invention provides a technique that allows the correspondence relationship between the state of an object and each frame image to be played back to be grasped easily.

### SUMMARY OF THE INVENTION

According to one aspect of the present invention, there is provided an image processing apparatus comprising: an extraction unit configured to extract at least one region of interest with respect to each frame image of a series of frame images of an object; a calculation unit configured to calculate a feature amount of the region of interest; and a display control unit configured to cause a display unit to display the feature amount as a time series graph, wherein when at least some of the series of frame images are to be played back as a moving image, the display control unit causes an index which represents a position corresponding to the displayed frame image to be displayed on the graph.

According to another aspect of the present invention, there is provided an image processing apparatus comprising: an image obtainment unit configured to obtain a series of frame images of an object; an obtainment unit configured to obtain state data which indicates a state of the object and generation time information of the state data; and a display control unit configured to cause a display unit to display the state data as a time series graph based on the generation time information, wherein when at least some of the series of frame images are to be played back as a moving image, the display control unit causes an index which represents a position corresponding to the displayed frame image to be displayed on the graph.

According to another aspect of the present invention, there is provided an image processing method comprising: extracting at least one region of interest with respect to each frame image of a series of frame images of an object; calculating a feature amount of the region of interest; and controlling to cause a display unit to display the feature amount as a time series graph, wherein when at least some of the series of frame images are to be played back as a moving image, an index which represents a position corresponding to the displayed frame image is displayed on the graph in the controlling.

According to another aspect of the present invention, there is provided an image processing method comprising: obtaining a series of frame images of an object; obtaining state data which indicates a state of the object and generation time information of the state data; and controlling to cause a display unit to display the state data as a time series graph based on the generation time information, wherein when at least some of the series of frame images are to be played back as a moving image, an index which represents a position corresponding to the displayed frame image is displayed on the graph in the controlling.

According to another aspect of the present invention, there is provided a non-transitory computer-readable storage medium storing a program to cause a computer to execute an image processing method comprising: extracting at least one region of interest with respect to each frame image of a series of frame images of an object; calculating a feature amount of the region of interest; and controlling to cause a display unit to display the feature amount as a time series graph, wherein when at least some of the series of frame images are to be played back as a moving image, an index which represents a position corresponding to the displayed frame image is displayed on the graph in the controlling.

According to another aspect of the present invention, there is provided a non-transitory computer-readable storage medium storing a program to cause a computer to execute an image processing method comprising: obtaining a series of frame images of an object; obtaining state data which indicates a state of the object and generation time information of the state data; and controlling to cause a display unit to display the state data as a time series graph based on the generation time information, wherein when at least some of the series of frame images are to be played back as a moving image, an index which represents a position corresponding to the displayed frame image is displayed on the graph in the in the controlling.

Further features of the present invention will become apparent from the following description of exemplary embodiments (with reference to the attached drawings).

### BRIEF DESCRIPTION OF THE DRAWINGS

FIG. 1 is a view showing the arrangement of a radiation imaging system according to an embodiment;

FIG. 2 is a block diagram showing the hardware arrangement of a radiation imaging control apparatus according to the first embodiment;

FIG. 3 is a block diagram showing the functional arrangement of the radiation imaging control apparatus according to the first embodiment;

FIG. 4 is a view showing a GUI of a display/operation apparatus of the radiation imaging control apparatus according to the first embodiment;

FIG. 5 is a view showing a range designation analysis window of the radiation imaging control apparatus according to the first embodiment;

FIG. 6 is a view showing a range addition window of the radiation imaging control apparatus according to the first embodiment;

FIG. 7 is a view showing a playback range setting window of the radiation imaging control apparatus according to the first embodiment;

FIG. 8 is a flowchart showing the procedure of processing performed by the radiation imaging control apparatus according to the first embodiment;

FIGS. 9A to 9C are explanatory views of feature amount calculation of a lung field region according to the first embodiment;

FIGS. 10A and 10B are views each showing an example of graph display of radiation imaging control apparatus according to the first embodiment;

FIG. 11 is an example of a DICOM data structure according to the first embodiment;

FIG. 12 is a block diagram showing the hardware arrangement of a radiation imaging control apparatus according to the second embodiment;

FIG. 13 is a block diagram showing the functional arrangement of the radiation imaging control apparatus according to the second embodiment;

FIG. 14 is a flowchart of the procedure of processing performed by the radiation imaging control apparatus according to the second embodiment;

FIG. 15 is a block diagram showing the functional arrangement of a radiation imaging control apparatus according to the third embodiment;

FIG. 16 is a view showing a GUI of a display/operation apparatus of the radiation imaging control apparatus according to the third embodiment;

FIGS. 17A to 17C are views showing thumbnail display according to the third embodiment; and

FIG. 18 is a view showing a GUI of a display/operation apparatus of a radiation imaging control apparatus according to the fourth embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

<Arrangement of Radiation Imaging System>

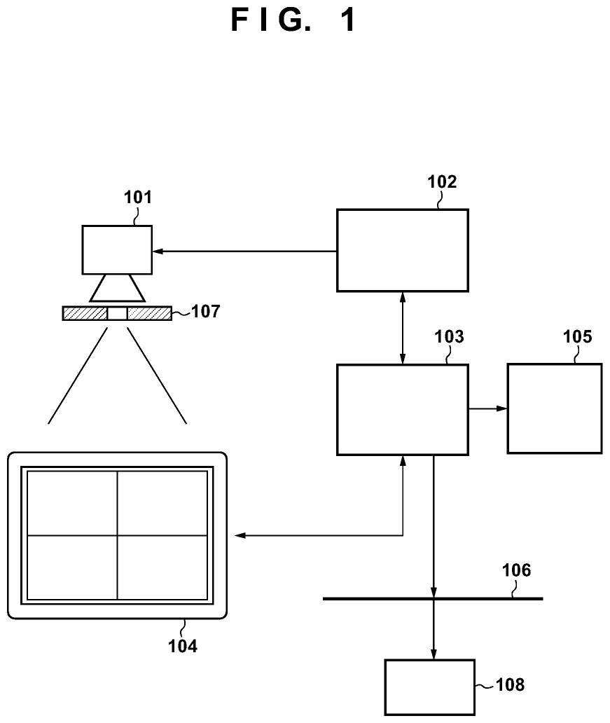

FIG. 1 is a view showing an example of the arrangement of a radiation imaging system according to the first embodiment. In the radiation imaging system, reference numeral 101 denotes a radiation generation apparatus; 102, a radiation control apparatus; 103, a radiation imaging control apparatus; 104, a radiation imaging apparatus; and 105, a display/operation apparatus. In addition, reference numeral 106 denotes an in-hospital network; 107, an aperture; and 108, an image server.

The radiation generation apparatus 101 is connected to the radiation control apparatus 102 and can generate radiation under a predetermined imaging condition set by the radiation control apparatus 102. The radiation control apparatus 102 sets the imaging condition of the radiation generation apparatus 101 and controls the operation of the radiation generation apparatus 101.

The radiation imaging control apparatus 103 controls the operation of the radiation imaging apparatus 104 and functions as an image processing apparatus that performs image processing on an image captured by the radiation imaging apparatus 104. The radiation imaging apparatus 104 obtains a moving image by detecting radiation transmitted through an object. The display/operation apparatus 105 can be operated by a user in addition to being display-controlled by the radiation imaging control apparatus 103 to display the state of the system and the obtained image.

The in-hospital network 106 is connected to the image server 108. A captured image output from the radiation imaging control apparatus 103 can be transferred to the image server 108 via the network 106. A radiologist will use a medical image management system called PACS (Picture Archiving and Communication Systems) to read out the image from the image server 108 and make a diagnosis. The aperture 107 is an aperture for defining the irradiation range of the radiation emitted from the radiation generation apparatus 101.

The image server 108 is a computer that includes a control unit, a storage unit, an operation unit, a display unit, a communication unit, and the like. An image DB (DataBase) is provided in the storage unit of the image server 108. The image DB stores image data transferred from the radiation imaging control apparatus 103.

<Hardware Arrangement>

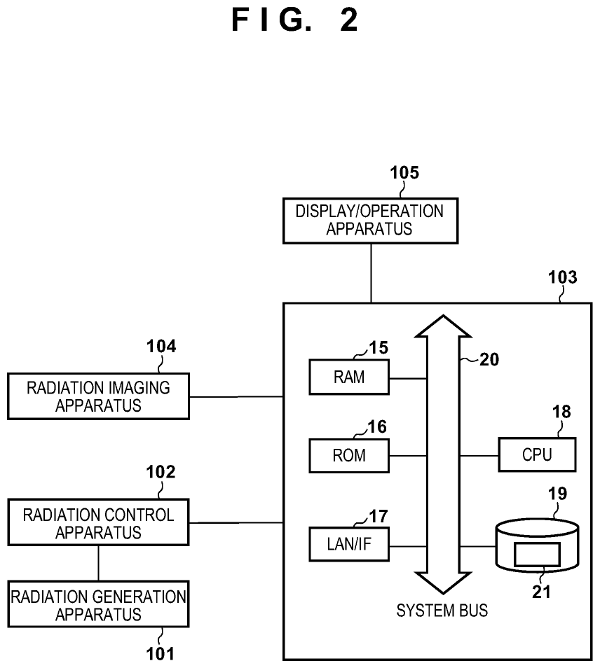

FIG. 2 is a block diagram which includes an example of the hardware arrangement of the radiation imaging control apparatus 103 according to the embodiment. The radiation imaging control apparatus 103 also functions as an image processing apparatus. The radiation imaging control apparatus 103 is connected to the radiation control apparatus 102, the radiation imaging apparatus 104, and the display/operation apparatus 105.

The radiation control apparatus 102 is connected to the radiation generation apparatus 101 via a cable and controls the radiation emitted by the radiation generation apparatus 101. The radiation generation apparatus 101 is implemented by, for example, a radiation tube, and irradiates an object (for example, a specific part of a patient) with radiation.

The display/operation apparatus 105 provides a GUI (Graphical User Interface) for integrally operating processing operations performed in the radiation imaging control apparatus 103 and displays obtained images. A display unit of the display/operation apparatus 105 is implemented by, for example, a monitor such a liquid crystal display or the like, and the display unit displays various kinds of information to an operator (a radiographer, a doctor, or the like). An operation unit of the display/operation apparatus 105 is implemented by, for example, a pointing device such as a mouse, a keyboard including a cursor key, numeric input keys, and various kinds of function keys, an operation button, and the like, and inputs various kinds of instructions from the operator to the radiation imaging control apparatus 103. Note that a touch panel integrating the display unit and the operation unit may also be used.

In addition, the radiation imaging control apparatus 103 is connected to the radiation imaging apparatus 104 via the cable, and a power supply voltage, an image signal, a control signal, and the like are exchanged between them via the cable. The radiation imaging apparatus 104 functions as an image capturing apparatus that detects the radiation transmitted through an object and obtains a radiation image based on the object. That is, radiation imaging is implemented when the radiation generation apparatus 101 and the radiation imaging apparatus 104 operate in cooperation. Note that the radiation imaging apparatus 104 is installed on an upright or supine imaging table.

The radiation imaging control apparatus 103 is formed by a RAM (Random Access Memory) 15, a ROM (Read Only Memory) 16, a LAN I/F 17, a CPU (Central Processing Unit) 18, an internal storage unit 19, a system bus 20, and the like.

The internal storage unit 19 is formed by a nonvolatile semiconductor memory, a hard disk, or the like, and stores a program 21. The RAM 15 stores the program 21 to be executed by the CPU 18, parameters required to execute processing by the program 21, or data such as processing results and the like. The ROM 16 is used as the main storage device of the CPU 18. In response to the operation of the display/operation apparatus 105, the CPU 18 reads out the program 21 stored in the internal storage unit 19, loads the program into the RAM 15, and executes, in accordance with the loaded program, the operation of each processing unit according to this embodiment.

The LAN I/F 17 is a communication unit that functions as an interface with a LAN (Local Area Network). The LAN I/F exchanges data with the image server 108 connected to the network 106 that has been built in the hospital.

<Functional Arrangement>

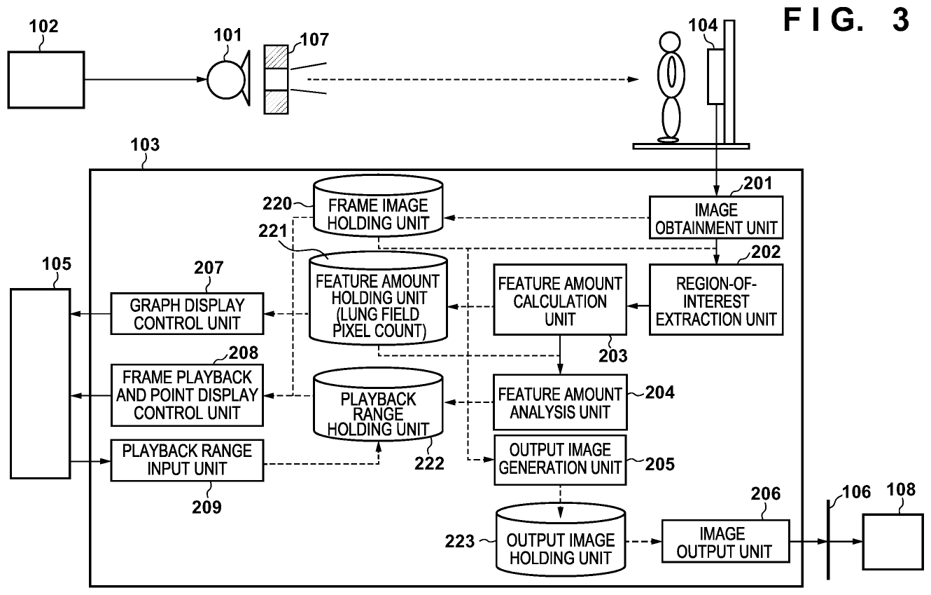

FIG. 3 is a block diagram showing an example of the functional arrangement of the radiation imaging control apparatus 103 according to this embodiment. The radiation imaging control apparatus includes an image obtainment unit 201, a region-of-interest extraction unit 202, a feature amount calculation unit 203, a feature amount analysis unit 204, an output image generation unit 205, an image output unit 206, the graph display control unit 207, and a frame playback and point display control unit 208. The radiation imaging control apparatus also includes a playback range input unit 209, a frame image holding unit 220, a feature amount holding unit 221, a playback range holding unit 222, and an output image holding unit 223.

The image obtainment unit 201 obtains a series of radiation frame images detected by a radiation detection unit (not shown) inside the radiation imaging apparatus 104, and stores the obtained series of images in the frame image holding unit 220. Note that device dependent offset correction, correction of shading generated due to a positional relationship, correction due to the system, and the like in a frame image may be performed in the radiation imaging apparatus 104 or in the image obtainment unit 201.

The region-of-interest extraction unit 202 extracts a region of interest by performing image processing on at least one specific part (region) of interest among a lung field region, a heart region, a pulmonary region, and a cardiovascular region, and the like. An example in which the lung field region is to be extracted as a part of interest will be described in this case. For example, the lung field region can be extracted by generating an image in which the edge of the frame image has been enhanced, and tracing the edge from a differential image in a spatial direction of the concentration.

The feature amount calculation unit 203 calculates feature amounts of an area of an extracted part of interest (region of interest), an average pixel value of the part of interest, a length of the part of interest, a concentration of a part of interest, and the like. In this example, a pixel count will be obtained as an amount corresponding to the area of the lung field region, and made to correspond with the frame number, and stored in the feature amount holding unit 221.

The feature amount analysis unit 204 analyzes, with respect to the feature amounts of all of the series of frame images obtained in the moving image capturing operation, the features of the movement of the lung field of based on the periodicity and the amplitude of the amount of change in the feature amounts in time series, determines the temporal range to be played back, and stores the range in the playback range holding unit 222.

The graph display control unit 207 displays, on the display/operation apparatus 105, a graph showing the relationship between the frame number and the feature amount obtained from the feature amount holding unit 221.

The frame playback and point display control unit 208 obtains, from the playback range holding unit 222, information of each frame range to be played back, obtains the corresponding frame image from the frame image holding unit 220, and displays the obtained frame image on the display/operation apparatus 105. In addition, the frame playback and point display control unit performs point display of the position of the corresponding frame number on the graph by using an index. The frame playback and point display control unit 208 performs processing in accordance with the frame rate so as to make the playback range loop. Note that the playback range holding unit 222 can hold a plurality of playback ranges. In a case in which the display/operation apparatus 105 has a GUI that displays the playback of only one playback range, a flag that indicates the playback range to be displayed will be provided.

The display/operation apparatus 105 can display a graph of the feature amounts of the region of interest, play back a moving image of the frame images of the playback range, and change the playback range on the graph in accordance with the operation by the user. The playback range input unit 209 obtains the information of the playback range input on the display/operation apparatus 105 in accordance with the operation by the user, and updates the information held in the playback range holding unit 222. The information related to the switching of the display playback range can be simultaneously input in addition to the information of the playback range in the playback range input unit 209, and these pieces of information are held in the playback range holding unit 222. The frame playback and point display control unit 208 controls, based on the information held in the playback range holding unit 222, the display/operation apparatus 105 so that moving image playback of the frame images and point display of each corresponding frame image on a graph will be performed.

After the playback range has been confirmed and the radiographer has pressed an “inspection end” button (not shown), the output image generation unit 205 will read out, from the frame image holding unit 220, each frame image of the playback range, and output the frame image to the output image holding unit 223. The image output unit 206 will output the frame images to the image server 108 via the in-hospital network 106.

<Example of GUI of Display/Operation Apparatus>

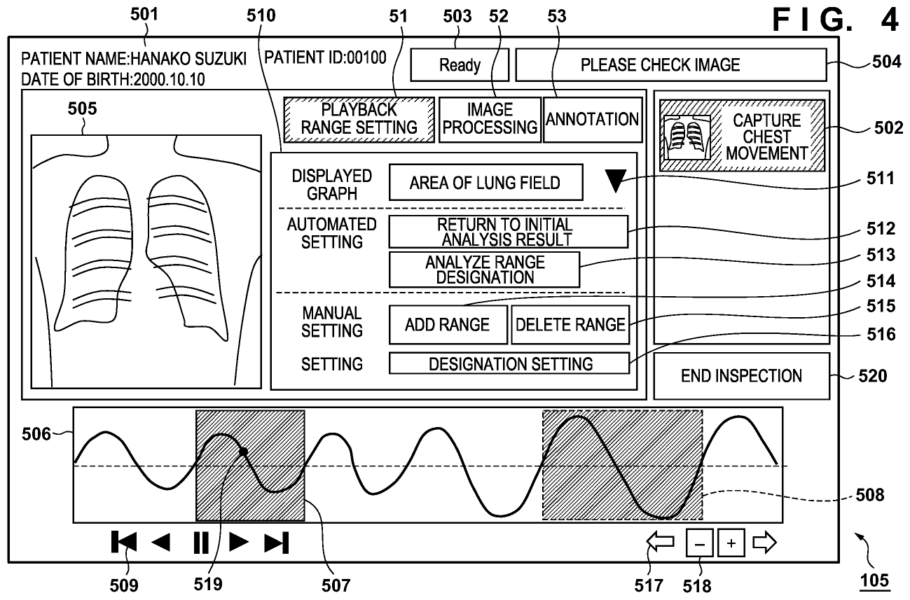

Next, FIG. 4 shows an example of a GUI to be displayed on the display/operation apparatus 105. Reference numeral 501 denotes a patient information display region; 502, a protocol display region; 503, a system state display region; and 504, a message display region. In addition, a reference numeral 505 denotes a frame image playback region; and 510, a processing region for performing various kinds of processing on a playback image. A graph display region 506 is a region for a time series graph of feature amounts. Reference numerals 507 and 508 each denote a frame image playback range on the graph. In this example, the frame image playback range 507 is in a selected state, and shows the playback range of frame images that are played back in a loop in the frame image playback region 505. On the other hand, although the frame image playback range 508 is not selected and is not being played back in the frame image playback region 505, it can be played back by, for example, touch and selecting the corresponding region on the graph. Also, the frame image playback range 507 can be expanded/reduced and moved by touch operation in a paused state that is set by pressing a pause button which is one of playback operation buttons 509. In addition, a graph that cannot fit into the graph display region 506 can be confirmed by operating a move button 517. An enlarge/reduce button 518 is a button for enlarging or reducing the graph in a horizontal direction.

Also, the playback operation buttons 509 are formed by a group of buttons for, from the left, moving to the first frame, performing reverse playback, pausing, performing playback, and moving to the final frame. Note that a playback frame position 519 is an index indicating a playback frame position corresponding to the frame image being played back in the frame image playback region 505.

In a processing region 510, the display contents of the processing region can be switched by a playback range setting tab 51, an image processing tab 52, and an annotation setting tab 53, and a state in which the playback range setting tab 51 is selected is shown in this case. The processing region 510 in a state in which the playback range setting tab 51 is selected is largely divided into three regions, and is divided into, from the top, a region for setting an analysis target part, a region for setting an automatic playback range, and a region for setting a manual playback range. A dropdown list 511 is set so that a part of interest can be selected. A button 512 for returning to an initial analysis result is a button to make the playback range return to the initial analysis result. A range designation analysis button 513 is a button for displaying a range designation analysis setting window which is used to set an analysis range on the graph.

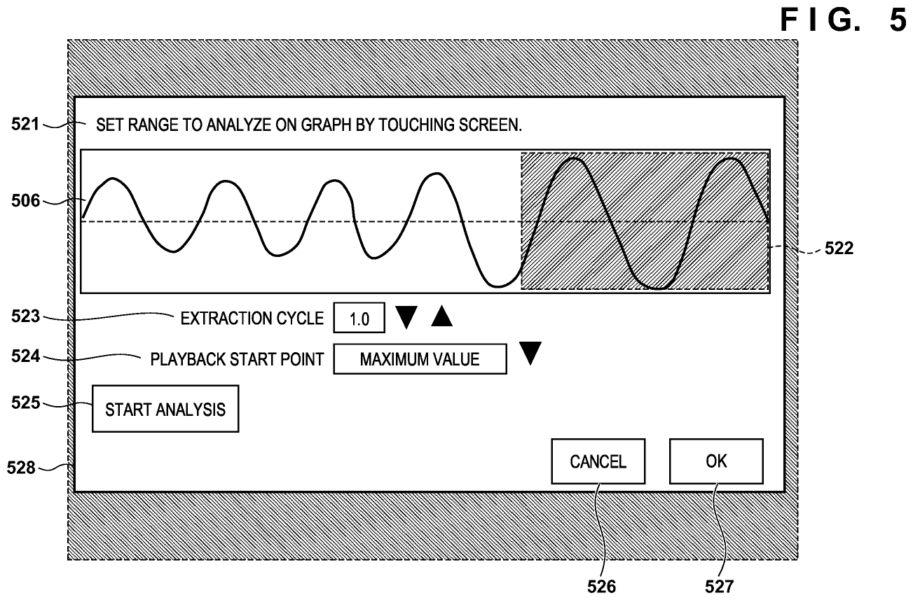

In this case, FIG. 5 is a view showing an example of the range designation analysis setting window, and an analysis can be performed after the designation range has been set. The range designation analysis button 513 of FIG. 4 can be pressed to call a range designation analysis setting window 528.

Reference numeral 521 is a message display region. An analysis range 522 indicated by a dotted line on the graph display region 506 can be set. An extraction cycle (one cycle in the example shown in FIG. 5) is set in an input region 523, a playback start point (starting from the maximum value in the example shown in FIG. 5) is set in an input region 524, and an analysis is started when an analysis start button 525 is pressed. The playback range of the analysis result is indicated by a solid line. Expansion, reduction, and movement of the analyzed playback range can be performed by touch operation in a manner similar to FIG. 4. When the playback range is confirmed, an OK button 527 can be pressed to return to the screen of FIG. 4, and the set playback range will be added to the graph display region 506. Note that CANCEL button 526 can be pressed to cancel the setting and to return to the screen of FIG. 4.

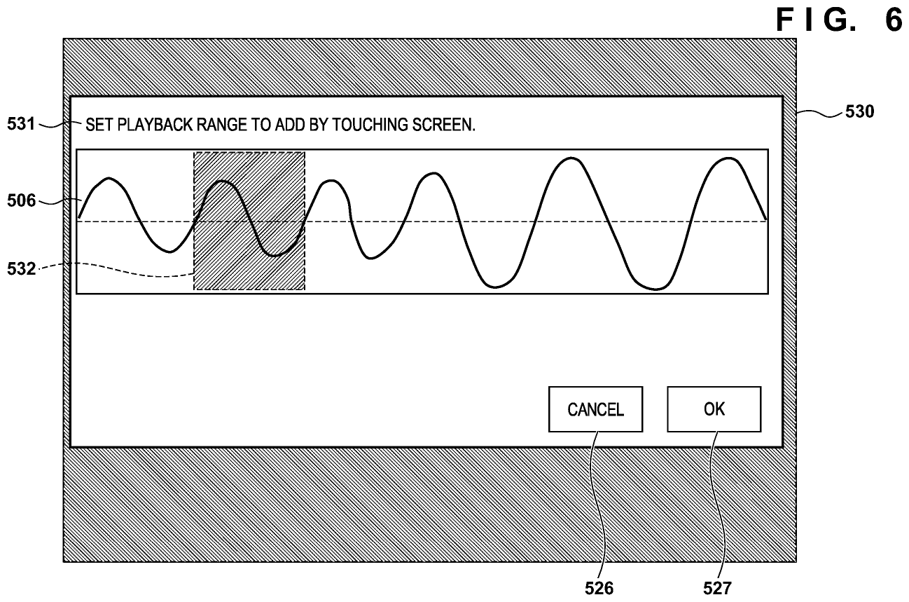

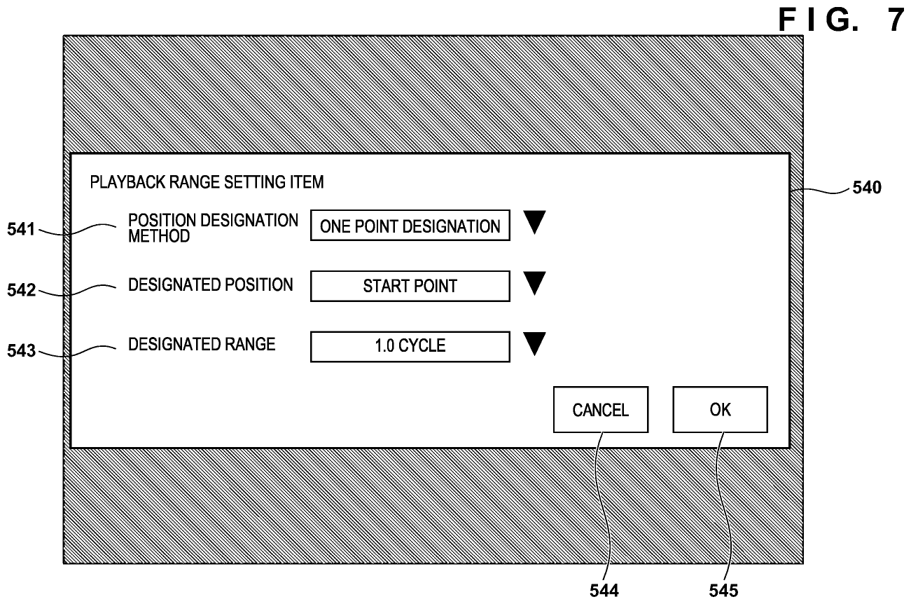

In addition, settings for manual analysis can be performed by using, as shown in FIG. 5, a range addition button 514, a range deletion button 515, and a designation setting button 516. A range addition window (FIG. 6) opens in response to the pressing of the range addition button 514, and settings can be made in this window. Furthermore, a range designation window (FIG. 7) opens in response to the pressing of the designation setting button 516, and settings can be made in this window.

The range deletion button 515 can be pressed to delete the playback range after touching and selecting the playback range to be deleted.

Next, FIG. 6 shows an example of a GUI of a playback range addition window 530 that opens in response to the pressing of the range addition button 514. Reference numeral 531 is a message display region. When the OK button 527 is pressed after designating and confirming the playback range by a touch operation, the screen returns to the screen of FIG. 4. Subsequently, the set playback range is added to the graph display region 506. Note that the settings can be canceled by pressing the CANCEL button 526, and the screen will return to the screen of FIG. 4.

In addition, FIG. 7 is an example of a GUI of a playback range setting window 540 that opens in response to the pressing of the designation setting button 516. Reference numeral 541 is a position designation method input region, and this input region accepts the setting as to whether to designate a position by using a single point or by using two points of the start point and the end point.

In the case of designation by a single point, an input region 542 for further setting a designation position will accept setting as to which of the start point, the midpoint, and the end point will be set as the single point. A designation range input region 543 will accept the designation of a playback range. Subsequently, when an OK button 545 is pressed after the confirmation, the screen returns to that of FIG. 4. Note that a CANCEL button 544 can be pressed to cancel the settings and to return to the screen of FIG. 4.

<Imaging Procedure>

The procedure of processing performed when a radiation image is to be captured in accordance with the sequence of the inspection by the radiation imaging system shown in FIG. 3 will be described.

First, patient information and inspection information are input to the radiation imaging control apparatus 103 based on an inspection request form or an inspection request from an RIS (Radiology Information System) (not shown) via the network. The patient information includes pieces of information such as the name of the patient, the patient ID, and the like, and the inspection information includes the imaging information defining the contents of the imaging to be performed on the patient. In this example, imaging information for performing imaging of a respiratory movement is included as the inspection information.

The input patient information and imaging information are displayed on the display/operation apparatus 105. The radiographer views the patient information displayed on the display/operation apparatus 105 and calls the corresponding patient into the imaging room. The radiographer presses an “imaging start” button (not shown) to prepare the radiation imaging apparatus 104 for imaging, and positions the patient so that imaging can be performed correctly. When the imaging preparation of the radiation control apparatus 102 and the radiation imaging apparatus 104 has been completed, the radiographer steps on a footswitch (not shown) to perform moving imaging capturing of the respiration of the patient. At this time, instructions may be output via a nurse call system to navigate the respiratory state of the patient by asking the patient to first breathe normally and then to take a deep breath next.

When the radiographer steps on the footswitch, radiation irradiation is performed continuously from the radiation generation apparatus 101, and the radiation transmitted through the object is detected by the radiation imaging apparatus 104 and obtained as image data by the radiation imaging control apparatus 103. Since the radiation imaging apparatus 104 obtains, as a single frame, image data at a predetermined interval such as 20 FPS, a moving image can be obtained. When the imaging of a desired respiratory state has been completed, the radiographer releases his/her foot from the footswitch to end the imaging operation.

<Processing (Graph Display of Lung Field Area)>

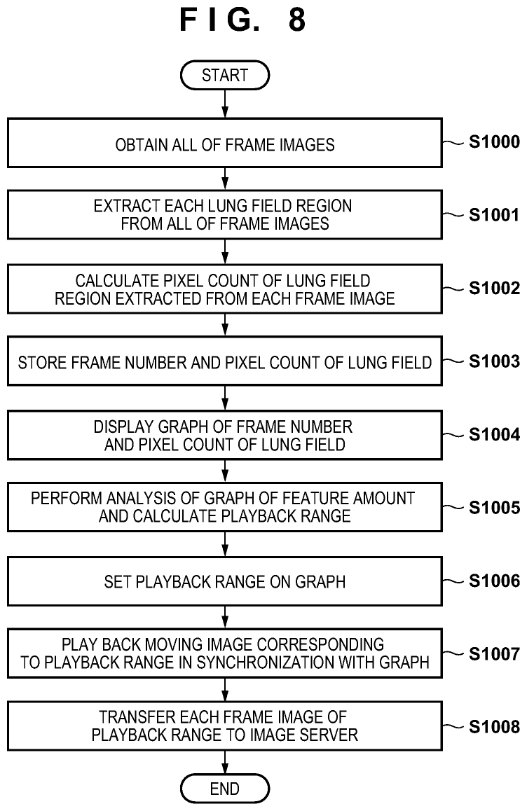

Image processing executed by the radiation imaging control apparatus 103 after the completion of imaging will be described with reference to FIG. 8.

In step S1000, the image obtainment unit 201 obtains all of the frame images obtained by the radiation imaging apparatus 104. In step S1001, the region-of-interest extraction unit 202 extracts each lung field region from all of the frame images. In step S1002, the feature amount calculation unit 203 calculates the pixel count corresponding to the area of each extracted lung field region.

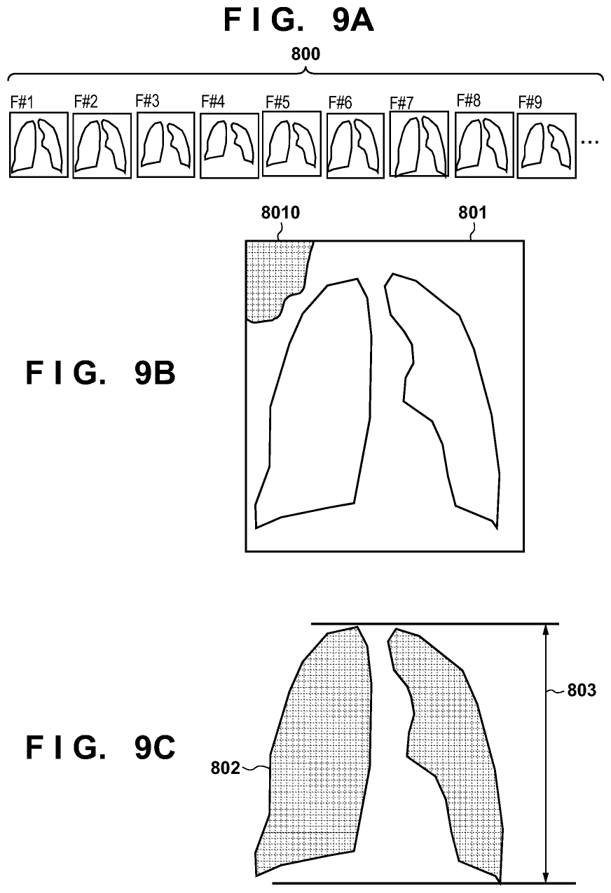

In FIG. 9A, reference numeral 800 denotes all of the frame images, and a symbol F #1 or the like indicates the frame number written above each frame image. In addition, FIG. 9B conceptually shows a frame image 801 and pixels 8010 forming the frame image 801. FIG. 9C shows a pixel count 802 of the lung field region. Note that a length 803 is the length in the longitudinal direction of the lung field region. For example, the contour of the lung field can be extracted by generating a differential image in the spatial direction of the concentration upon enhancing the contour of the lung field by performing edge enhancement by performing image processing on the obtained frame image 801. Subsequently, the pixel count 802 of the extracted lung field region can be calculated to obtain a feature amount proportional to the area of the lung field.

In step S1003, the feature amount calculation unit 203 calculates the feature amount (lung field pixel count) of each of all of the frame images, and stores the feature amount in association with the corresponding frame number or imaging time. In step S1004, the graph display control unit 207 displays, on the graph display region 506 of FIG. 4, a graph of the feature amount with respect to each frame number.

In step S1005, the feature amount analysis unit 204 performs an analysis of the period and the amplitude of the graph displayed in the graph display region 506. In relation to the period, the frequency may be obtained by FFT (Fast Fourier Transform) or the maximum interval and the minimum interval of the amplitude may be calculated. As a result, two types of waveforms representing normal respiration and deep respiration are extracted, and a playback range is automatically extracted for each waveform by using the setting value of a playback start point and the setting value of an extraction cycle. Note that the setting value of the playback start point can be set by using the same GUI as that of input region 524 for the playback start point, and the extraction cycle can be set by using the same GUI as that of the input region 523 for the extraction cycle shown in FIG. 5.

In step S1006, the frame playback and point display control unit 208 superimposes and displays, on the graph display region 506, each playback region extracted by the analysis. In step S1007, the frame playback and point display control unit 208 plays back a moving image corresponding to the playback range in synchronization with the graph. More specifically, each movement frame image belonging to the set playback range is displayed (is played back as a moving image) in the frame image playback region 505, and an index that indicates the playback frame position 519 corresponding to the frame image which is being played back in the frame image playback region 505 is displayed in the graph display region 506. As a result, the correspondence relationship between the state of the object and each frame image played back as a moving image can be grasped easily. The radiographer can change/correct the playback range by confirming the playback frame position 519. The index indicating the playback frame position 519 is at, for example, a position included in the playback range. When the setting of the playback range is completed, the radiographer presses an “imaging end” button (not shown) to end the imaging operation.

In step S1008, each movement frame image of the playback range is generated by the output image generation unit 205. Subsequently, the image output unit 206 transfers each movement frame image of the playback range to the image server 108. As a result, a doctor can read out only the frame images belonging to the playback range from the image server 108 to perform radiographic interpretation.

As described above, in this embodiment, at least one region of interest will be extracted with respect to each frame image of a series of frame images of an object, and the calculated feature amounts will be displayed as a time series graph. Subsequently, when the series of frame images are to be at least partially played back as a moving image, an index indicating a graph position corresponding to the displayed frame image will be displayed.

As a result, the correspondence relationship between the state (for example, the expiration state or the inspiration state) of the object and each frame image that is played back as a moving image can be grasped easily. Hence, in a case in which, for example, the lung field region is the region of interest of the currently displayed frame image that is being played back as a moving image, it becomes possible to easily grasp to which inspiration or expiration timing the frame image corresponds.

In addition, in this embodiment, it is arranged so that each playback range will be superimposed on the graph and the playback range can be changed based on a user operation. Hence, the user, who has seen the correspondence relationship between the state (for example, the expiration state or the inspiration state) of the object and each frame image that is played back as a moving image, can easily change the playback range of the moving image to make the range more suitable for diagnosis.

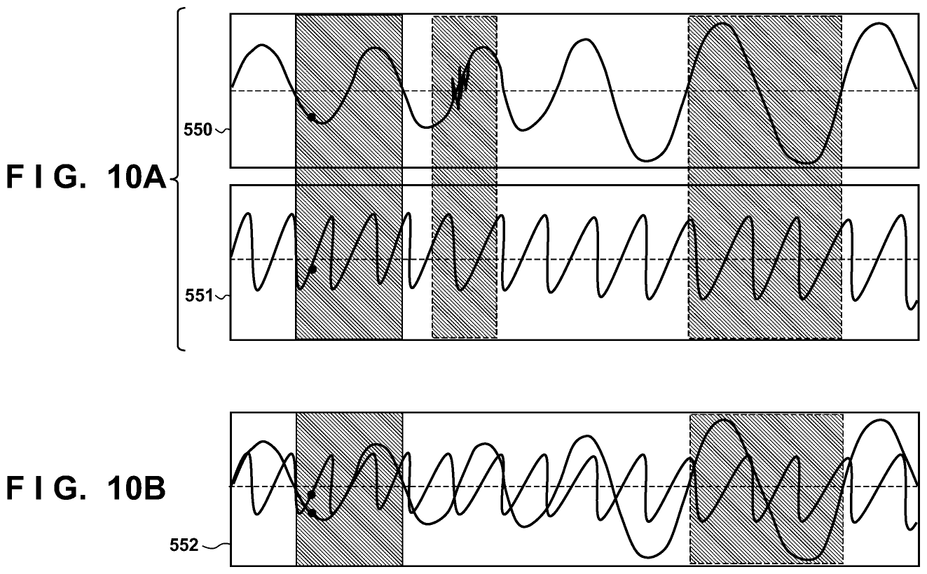

Note that although a normal respiratory state is shown in the example of the graph which shows a change in the size of the lung field and is displayed in the graph display region 506, a graph as displayed in a graph display region 550 of FIG. 10A will be shown in a case in which a respiration irregularity has occurred. Thus, a graph in which a large frequency irregularity is superimposed on the respiration amplitude is formed in this manner. The radiographer can, for example, correct the range which includes the irregularity as a playback range to obtain a moving image of a playback range which is more suitable for diagnosis.

In addition, peaks will appear in three types of frequencies when FFT is performed, and a normal respiratory cycle, a deep respiratory cycle, and a range in which a respiration irregularity has occurred may be automatically set as playback ranges. Note that a graph display region 551 of FIG. 10A will be described later.

<Graph Display of Area Other Than Lung Field Area>

Although the flowchart of FIG. 8 described an example in which a graph of the change in the area of the lung field region is generated by counting the pixel count of the lung field region, the length in the longitudinal direction of the lung field may be calculated as a feature amount. In such a case, the length 803 shown in FIG. 9C will correspond to the length in the longitudinal direction of the lung field, and a pixel count corresponding to the length 803 will be the feature amount. In this case as well, moving image playback can be performed by displaying the pixel count of the length corresponding to each frame number as the feature amount on a graph, analyzing the period and the amplitude of the graph by the feature amount analysis unit 204, and setting and showing a playback range on the graph.

Alternatively, in FIG. 9B, the heart region may be extracted in addition to the lung field to calculate an amount proportional to the area of the heart, and graph display, graph analysis, and setting of the playback range can be performed in a similar manner. In this case, the heart region may be used as reference to determine the playback range of the movement of the lung field or the playback range may be determined to observe the movement of the heart.

In the former case, the graph of the lung field region and the graph of the heart region are vertically arranged in the manner of the graph display region 550 and the graph display region 551 as shown in FIG. 10A. In this case, the playback range is set by using the analysis result of the graph of the lung region.

On the other hand, in the latter case, the graph of the lung field region and the graph of the heart region are displayed by changing the colors of the respective graphs and overlaying the graphs as shown in a graph display region 552 of FIG. 10B. A more suitable lung field playback range can be set by observing the movement of the heart.

For example, although there are sports athletes who have a long cardiac cycle interval, a playback range that does not include one cardiac cycle may not be a playback range that is suitable for diagnosis. Hence, by expanding, based on an operation by the user, the playback range to include at least one cardiac cycle, the moving image of a playback range which is more suitable for diagnosis can be extracted. In this case, a plurality of cycles can be included in the respiratory cycle of the lung field region.

Since the latter case is for observing the movement of the heart, the heart region of each frame image is trimmed as a partial image. Trimming may be performed in accordance with an instruction from the operator or the trimming size may be automatically determined based on the maximum region of a result extracted from the heart region. Loop playback of the playback range is performed for the trimmed region.

In addition, the playback region may be set based on the difference in the movement of the left and right lung fields. In general, the amount of movement of the left lung is larger than that of the right lung, and the amount of movement tends to be proportional to this movement. However, if there is a location that is not in correspondence with this kind of movement, the location will be confirmed by a playback operation. Hence, the graph of the left lung region and the graph of the right lung region may be vertically displayed or displayed by changing the colors and overlaying the respective graphs.

Furthermore, the volume of the blood flow in the lung field may be extracted as a feature amount to generate a graph. Since the volume of blood flow in the lung field and the pixel concentration (pixel value) have a proportional relationship, a region in which the volume of blood flow in the lung field can be observed will be extracted, and a graph showing the increase/decrease in the volume of the blood flow can be generated and displayed based on the average pixel value of this region. A more suitable playback range can be set by aligning and displaying the graph of the lung field region and the graph of the increase/decrease in the volume of blood flow.

It can be arranged so that these graphs can be switched by using the dropdown list 511 of FIG. 4. Also, although it is shown that a graph can be switched and displayed by using the dropdown list 511 in the example of FIG. 4, the playback range may be switched by using a tab. Furthermore, a plurality of the frame image playback regions 505 may be provided to simultaneously play back a plurality of movement frame images.

<Manual Playback Range Setting>

In this embodiment, the initial value of a playback range is set by using the analysis result of the feature amount analysis unit 204, and a moving image of this playback range is played back. However, the present invention is not limited to this. Only the graph of the graph display region 506 may be displayed first by the display/operation apparatus 105 without executing analysis by the feature amount analysis unit 204, and the playback of the moving image frame may be performed in the frame image playback region 505 after confirming the playback range in the graph display region 506.

<Time Series Data Output>

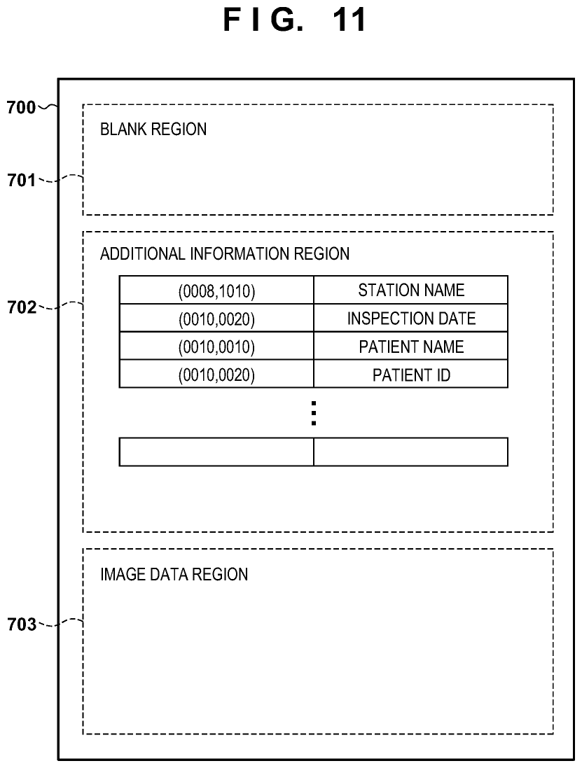

In this embodiment, the frame images of the playback range are transferred to the image server in step S1008. At this time, the frame images are transferred as a file having a data structure based on the DICOM (Digital Imaging Communications in Medicine) standard. As shown in FIG. 11, a DICOM image file is largely formed by an image data region 703, a blank region 701 of a header portion, and an additional information region 702.

The additional information region 702 of a DICOM image file is formed by an aggregate of data elements, and each data element includes standard tags (a group number and an element number) and the corresponding tag information (data length and data). The tag information is various kinds of attribute information related to the image data such as the patient information, the imaging condition information, the image information, the display information, and the like. The patient information includes personal information for specifying the patient such as the name of the patient, the patient ID, the date of birth, and the like. The imaging condition information includes the image type such as a first obtained image, a second obtained image, and the like, the imaging part, information related to the imaging state such as the radiation current value and the voltage value at the time of imaging, and the like. The image information includes information indicating the inspection ID, the type of the medical diagnosis apparatus, and the like. The display information includes information such as the contrast of the image, the alignment order, the arrangement, and the series number (the group ID of the image) of the image, and the like. The file code of each DICOM image file and the like can be included as other information. In addition, it is possible to set, other than the standard tags, a private tag that can be interpreted by only specific apparatuses.

The frame images of the playback range are included in the image data region 703, attached with the additional information region 702 and a blank region 700, and transferred as a DICOM image file.

At this time, by including the time-series feature amount data in the private tag of the additional information region 702 so that the data can be displayed on the side of the PACS, the doctor will be able to make a diagnosis based on a graph showing the movement state. As a result, it will allow the doctor to make a diagnosis more accurately.

### Second Embodiment

The first embodiment described an example in which the feature amounts of regions of interest such as the lung field, the heart, the volume of blood flow of the lung field, and the like are calculated based on a series of frame images, the calculated feature amounts are displayed on a graph, and a position corresponding a frame image that is being played back as a moving image is displayed as a point on the graph. In contrast, the second embodiment will not describe an example in which the feature amounts of regions of interest are displayed on a graph, but will describe an example in which state data obtained from actual measurement of an object is displayed on a graph.

Since the arrangement of a radiation imaging system according to this embodiment is the same as that shown in FIG. 1 of the first embodiment, a description thereof will be omitted.

<Hardware Arrangement>

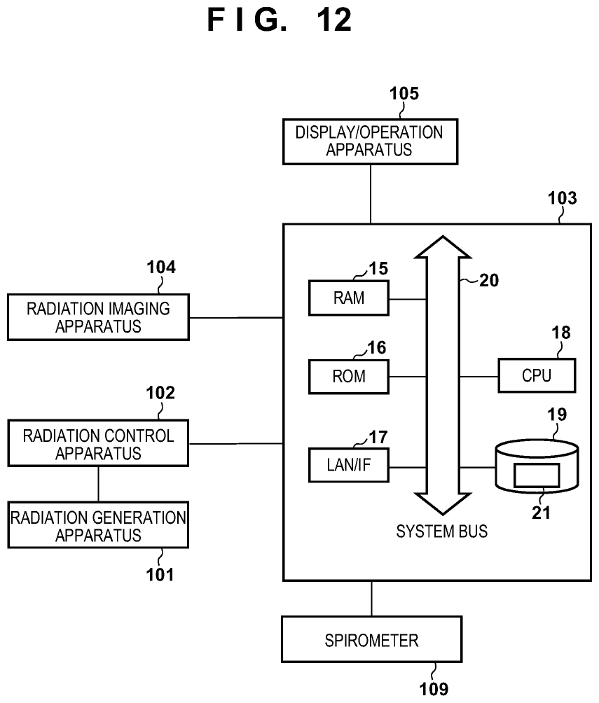

FIG. 12 is a block diagram including an example of the hardware arrangement of a radiation imaging control apparatus according to the second embodiment. In addition to the arrangement shown in FIG. 2, a spirometer 109 that measures the state data of an object during moving image capturing is connected to a radiation imaging control apparatus 103. Components other than this are similar to those of FIG. 2. The spirometer 109 is a measurement device used to inspect the respiratory function of the lung, and is a measurement device that measures the speed and the capacity of air that is exhaled and inhaled. A graph proportional to the area of the lung field can be generated by loading the measurement times and flow rates into the radiation imaging control apparatus 103 and arranging these pieces of information in time series. Note that although the state data can be obtained from the spirometer 109 in real time, if a device type that cannot transmit data in real time is to be used, association with each frame image may be performed by collectively receiving the state data later.

<Functional Arrangement>

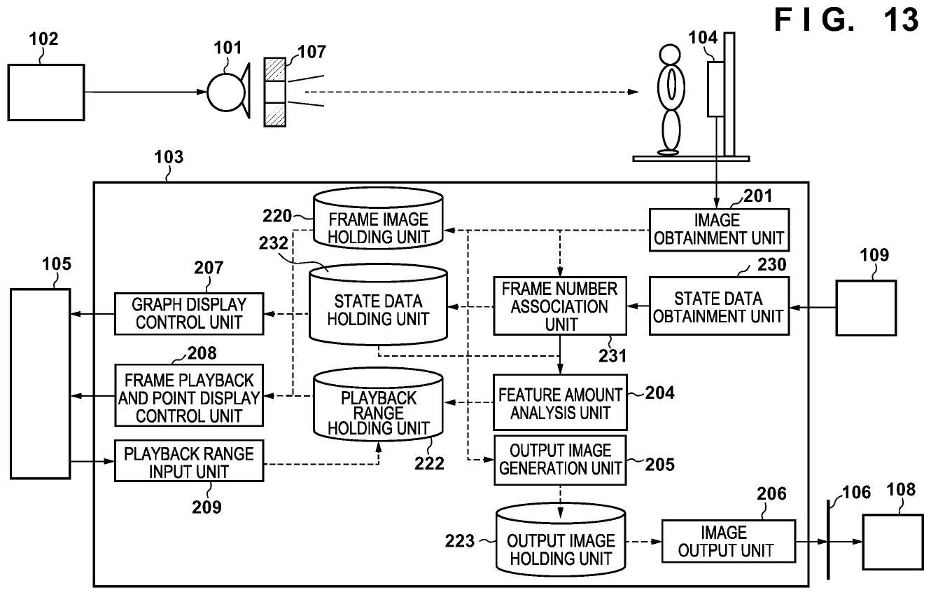

FIG. 13 is a block diagram including an example of the functional arrangement of the radiation imaging control apparatus according to the second embodiment. This arrangement differs from the arrangement of the radiation imaging control apparatus 103 shown in FIG. 3 in that a state data obtainment unit 230, a frame number association unit 231, and a state data holding unit 232 are included instead of a region-of-interest extraction unit 202, a feature amount calculation unit 203, and a feature amount holding unit 224. Different points will be mainly described hereinafter.

State data measured by the spirometer 109 is loaded into the radiation imaging control apparatus 103 from the state data obtainment unit 230, and the state data and each frame number are associated with each other by the frame number association unit 231. As an example, the frame number association unit 231 associates state data, which has a state data generation time closes to the frame image obtainment time in a radiation imaging apparatus 104, with a corresponding frame number. Alternatively, linear interpolation may be performed to calculate the state data of the frame image obtainment time. The state data that has been associated with the corresponding frame number is held by the state data holding unit 232. In this case, since the state data obtained from the spirometer 109 is a respiratory flow rate, the capacity of the lung can be obtained by obtaining the initial capacity and the integrals values up to the state data obtainment time. Subsequently, a graph representing the size of the lung field can be generated by arranging the integral values in time series. A feature amount analysis unit 204 will calculate the period and the amplitude of the graph to obtain the playback range in the same manner as in the first embodiment, and a playback range holding unit 222 will hold the generated playback range.

<Processing>

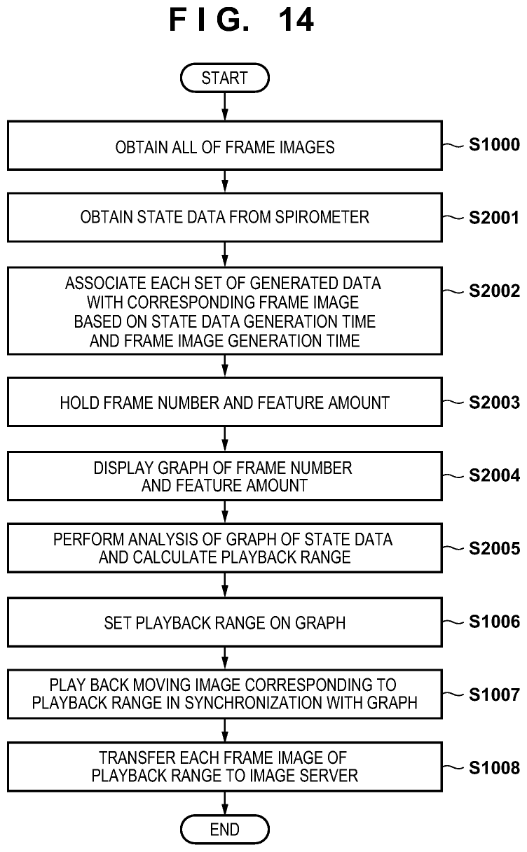

Image processing executed by the radiation imaging control apparatus 103 after the completion of imaging will be described next with reference to FIG. 14. The same processes as those of FIG. 8 are denoted with the same step numbers, and a detailed description thereof will be omitted.

In step S2001, the state data obtainment unit 230 obtains the state data measured by the spirometer 109. In step S2002, after obtaining generated data by converting the state data obtained from the spirometer 109 into feature amounts representing the area of the lung field, the frame number association unit 231 associates, based on the state data generation time and the frame image generation time, each set of converted generated data (feature amount) with a corresponding frame number.

In step S2003, the state data holding unit 232 holds the frame number and the feature amount of each frame image. In step S2004, the graph display control unit 207 displays a graph of the feature amount with respect to each frame number in a graph display region 506 shown in FIG. 4.

In step S2005, the feature amount analysis unit 204 analyzes the period and the amplitude of the graph displayed in the graph display region 506. The analysis method is the same as that used in the first embodiment, and a playback range is extracted automatically as a result. Note that the setting value of a playback start point will be set by using the same GUI as that of a input region 524 for the playback start point, and an extraction cycle can be set by using the same GUI as that of an input region 523 for the extraction cycle shown in FIG. 5. Subsequent processes are the same as those of the first embodiment.

As described above, the state data measured by a spirometer can be used to obtain the same effect as that of the first embodiment. In addition, a capnography device that measures the carbon dioxide included in the inspiration and the expiration may be used as the device for measuring the state of the object instead of the spirometer. The respiratory cycle can be understood because the capnography device will measure the concentration of carbon dioxide. Alternatively, a electrocardiograph may be used to check the pulsation of the heart.

### Third Embodiment

The third embodiment will describe an example which the suitability of a playback range can be determined more easily by displaying a thumbnail image of each frame image together with the graph.

Since the arrangement of a radiation imaging system according to this embodiment is the same as that shown in FIG. 1 of the first embodiment, a description thereof will be omitted. In addition, since the hardware arrangement according to this embodiment is the same as that shown in FIG. 2 of the first embodiment, a description thereof will be omitted.

<Functional Arrangement>

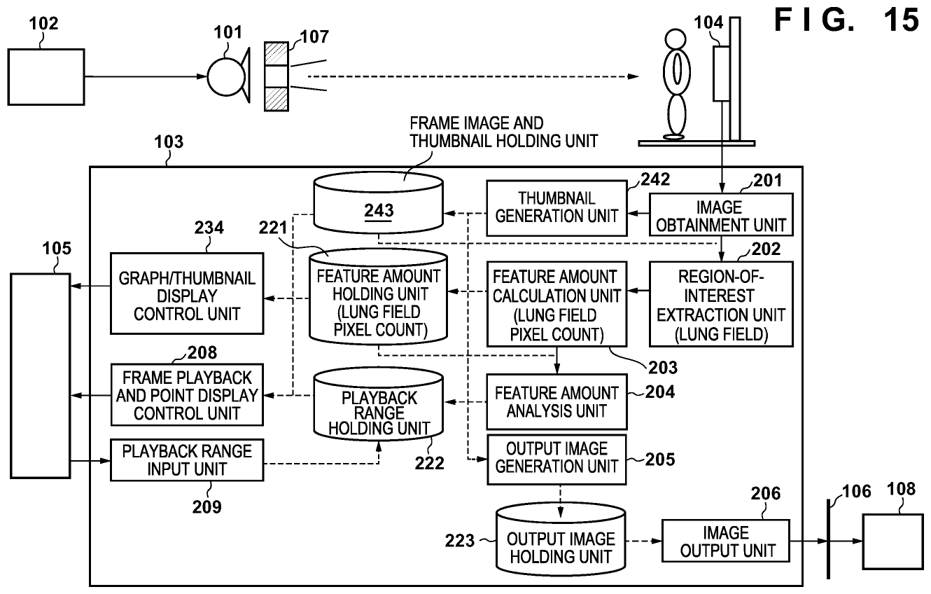

FIG. 15 is a block diagram including an example of the functional arrangement of a radiation imaging control apparatus according to the third embodiment. This arrangement differs from the arrangement of the radiation imaging control apparatus 103 shown in FIG. 3 in that a thumbnail generation unit 242, a frame image and thumbnail holding unit 243, and a graph/thumbnail display control unit 234 are included instead of a frame image holding unit 220 and a graph display control unit 207. Different points will be mainly described hereinafter.

The image obtainment unit 201 obtains a series of radiation frame images detected by a radiation detection unit (not shown) in a radiation imaging apparatus 104. An image obtainment unit 201 outputs the series of radiation frame images to the thumbnail generation unit 242.

The thumbnail generation unit 242 generates thumbnail images of the series of radiation frame images. Each thumbnail image is generated by thinning pixels and performing image compression such as JPEG (Joint Photographic Experts Group) compression or the like to reduce the storage capacity. The frame images obtained by the image obtainment unit 201 and the thumbnail images generated by the thumbnail generation unit 242 are held in the frame image and thumbnail holding unit 243.

A region-of-interest extraction unit 202 and a feature amount calculation unit 203 will perform image processing, feature amount calculation, and feature amount analysis on each frame image in the same manner as that in the first embodiment, and the obtained results are held in a feature amount holding unit 221 and a playback range holding unit 222. In this case, image processing, feature amount calculation, and feature amount analysis may be performed on, instead of each frame image, each thumbnail image generated by the thumbnail generation unit 242 to improve the processing speed.

The graph/thumbnail display control unit 234 will obtain, from the feature amount holding unit 221, a graph showing the relationship between the frame numbers and the feature amounts, display the obtained graph on a display/operation apparatus 105, obtain the corresponding thumbnail images from the frame image and thumbnail holding unit 243, and display the thumbnail images in correspondence with the graph. Since it is sufficient for the overview of a moving image to be known from each thumbnail image, each thumbnail image may be displayed at, for example, 2 FPS (0.5 sec interval) with respect to a moving image having a frame rate of 10 FPS.

<Example of Thumbnail Display>

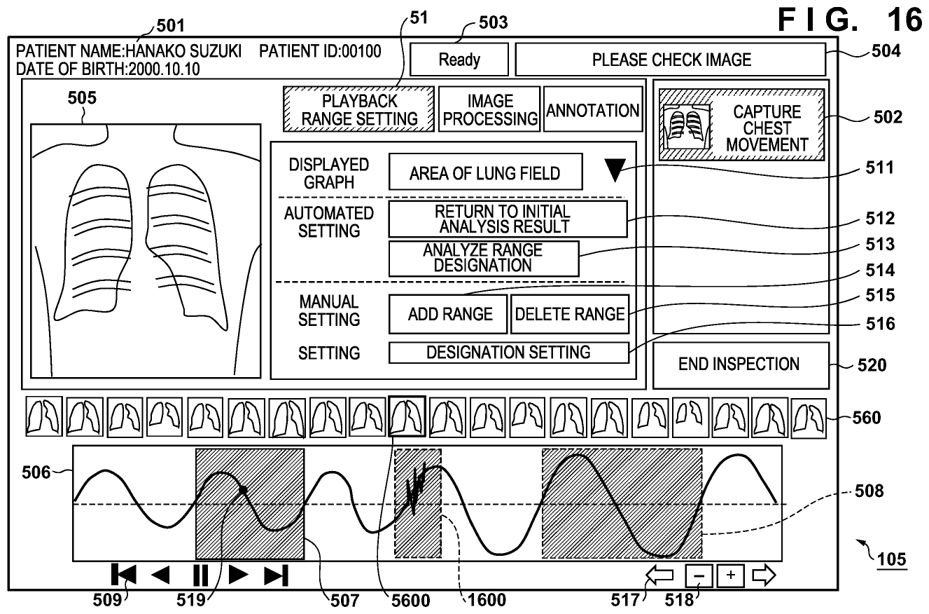

FIG. 16 is an example of a GUI which is displayed on the display/operation apparatus 105, and a series of thumbnail images are displayed in a region indicated by a thumbnail 560. Each thumbnail image is a thumbnail image of a frame image of a corresponding point on the graph. If a respiration irregularity is found, the corresponding thumbnail image is marked so that the image will be conspicuous. In this example, such a thumbnail is emphasized and displayed by surrounding the thumbnail with a frame 5600 which has a different color from the frames of other thumbnail images. Reference numeral 1600 denotes a playback range including the respiration irregularity.

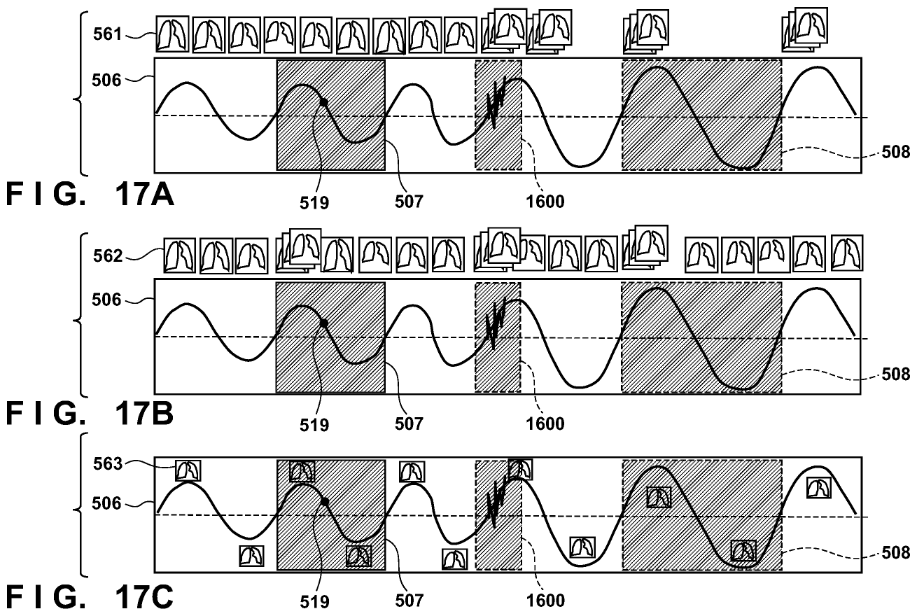

FIGS. 17A to 17C are views showing another example of thumbnail display. Although display was performed at a 0.5 sec interval in the thumbnail 560 because the region is limited, thumbnail display can be performed at a shorter interval by integrating unnecessary ranges.

A thumbnail 561 shown in FIG. 17A is an example in which thumbnail images of a playback range 507 are displayed at a shorter time interval (for example, a 0.25 sec interval) than the thumbnail 560, and only the thumbnail image of the starting point is shown in each range other than the frame image playback range 507. Since only the image of the start point is displayed in each range other than the playback range 507, an icon with overlapping thumbnail images will be shown. As a result, the thumbnail images of the playback range 507 can be confirmed in more detail. At this time, by displaying excessive portions outside the start and end points of the playback range 507 by about 10%, the excessive portions can be used as reference information when the playback range 507 is to be corrected.

In addition, a thumbnail 562 shown in FIG. 17B is an example in which the time interval between thumbnail images other than those belonging to the playback range 507, a playback range 508, and the playback range 1600 is shortened and displayed. An icon with overlapping thumbnail images is shown in each of the playback range 507, the playback range 508, and the playback range 1600 so as to save space. In addition, each thumbnail may be arranged on the graph as in the manner shown by a thumbnail 563 of FIG. 17C.

As described above, according to this embodiment, since a series of thumbnail images can be displayed together with a graph, the cycle of a region (the lung field or the like) of interest can be intuitively grasped, and it is possible to set a playback range suitable for diagnosis.

### Fourth Embodiment

Although an example in which the entire frame image is set mainly as the processing target has been described in each of the embodiments described above, the fourth embodiment will describe an example in which an image obtained by determining and trimming a partial region of the frame image is set as the processing target. Since the system arrangement and the apparatus arrangement are the same as the first embodiment, a description thereof will be omitted.

After a series of radiation frame images detected by a radiation detection unit (not shown) of a radiation imaging apparatus 104 and the series of images have been loaded into the radiation imaging control apparatus 103 by the image obtainment unit 201, a partial region is determined and trimmed. Subsequently, the trimmed image is used as the frame image.

The trimming method may be a method in which a user uses a captured image displayed on a display/operation apparatus 105 to instruct and determine a trimming range (partial region) by operating the display/operation apparatus 105 or may be a method in which a trimming range (partial region) is automatically determined based on an extraction result by a region-of-interest extraction unit 202.

This embodiment will describe an example in which a frame image of the movement of the lung field and a frame image of the movement of the heart are cut out from a frame image obtained by performing respiratory moving image capturing. The heart is at a center portion on the right side with respect to the image, and this portion will be trimmed, and the playback range will be played back as a moving image.

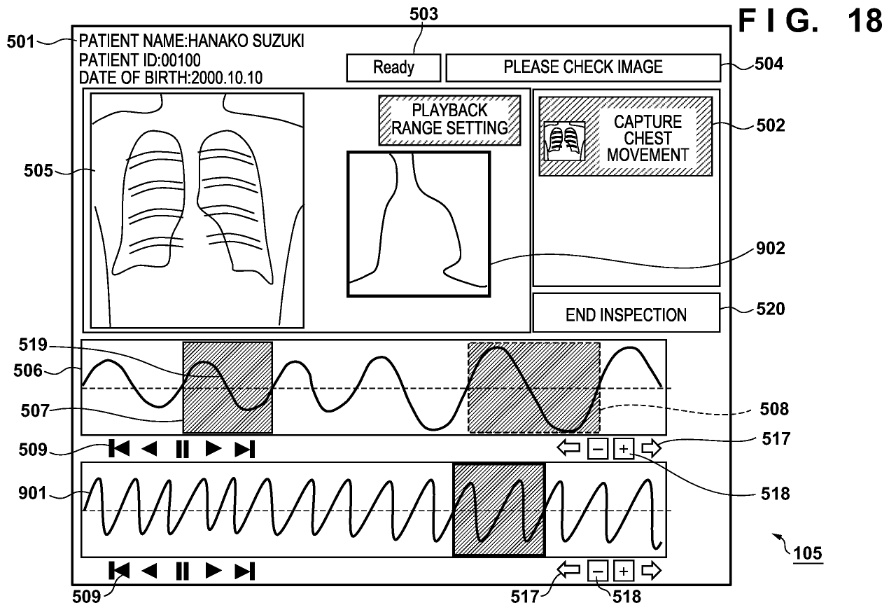

FIG. 18 shows an example of a GUI displayed on the display/operation apparatus 105. Reference numeral 901 is a graph display region in which a graph representing the size of the heart is displayed. Reference numeral 902 is a trimmed region playback region that plays back each trimmed image. The playback range of a frame image playback region 505 corresponds to the playback range set in a graph display region 506 which shows the size of the lung field. The playback range of the trimmed region playback region 902 of the moving image of the heart corresponds to the playback range on the graph displayed in the graph display region 901. In this manner, a plurality of parts can be arranged and displayed so that the movements of the plurality of parts can be confirmed simultaneously.

In addition, the data amount can be reduced by trimming the partial region. Also, it is effective for diagnosis to output one frame image as a still image together with the trimmed image to PACS by an image output unit 206 so that it will be possible to view PACS and know to which region of the frame image the trimmed region corresponds. In this case, it may be set so that it will be possible to designate a specific state, such as the maximal expiration state, the maximal inspiration state, a midpoint between the maximal expiration state and the maximal inspiration state, as the still image in accordance with the preference of the radiologist.

Note that although this embodiment described an example in which graphs of the feature amounts of the lung field and the heart as the two regions of interest are displayed, the present invention is not limited to this. For example, graphs of the feature amounts of the left lung field region and the right lung field region may be generated, and these two graphs may be arranged and displayed together with the frame images to be played back as a moving image.

As described above, according to this embodiment, the data amount can be reduced by setting, as a processing target, a trimmed image obtained by trimming a frame image. In addition, a plurality of parts can be arranged and displayed so that it will be easy to simultaneously confirm the movements of the plurality of parts.

According to the present invention, the correspondence relationship between the state of the object and each frame image to be played back as a moving image can be grasped easily.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-103946, filed Jun. 3, 2019, which is hereby incorporated by reference herein in its entirety.

What is claimed is:

1. An image processing apparatus comprising:
an extraction unit configured to extract at least one region of interest with respect to each frame image of a series of frame images of an object;
a calculation unit configured to calculate a feature amount of the region of interest; and
a display control unit configured to cause a display unit to display the feature amount as a time series graph,
wherein when at least some of the series of frame images are to be played back as a moving image, the display control unit causes an index which represents a position corresponding to the displayed frame image to be displayed on the graph.

2. The apparatus according to claim 1, wherein the region of interest is at least one of a lung field region, a heart region, a pulmonary region, and a cardiovascular region.

3. The apparatus according to claim 1, wherein the feature amount is one of an area of the region of interest, an average pixel value of the region of interest, a length of the region of interest, and a concentration of the region of interest.

4. The apparatus according to claim 1, further comprising:
a setting unit configured to set, on the graph, at least one temporal playback range for playing back at least some of the series of frame images as a moving image.

5. The apparatus according to claim 4, wherein the setting unit sets the playback range based on an operation by a user.

6. The apparatus according to claim 4, wherein in response to the selection of a tab, by an operation of a user, from not less than one tab corresponding to not less than one playback range, the setting unit sets a playback range corresponding to the tab.

7. The apparatus according to claim 4, wherein the index is displayed on the graph in the playback range.

8. The apparatus according to claim 1, wherein the display control unit causes a thumbnail image of the frame image to be displayed in time series on the graph.

9. The apparatus according to claim 1, further comprising:
a determination unit configured to determine a partial region which is a region of a part of the frame image,
wherein a frame image of the partial region is played back as a moving image.

10. The apparatus according to claim 9, further comprising:
an output unit configured to output the frame image and the frame image of the partial region.

11. An image processing apparatus comprising:
an image obtainment unit configured to obtain a series of frame images of an object;
an obtainment unit configured to obtain state data which indicates a state of the object and generation time information of the state data; and
a display control unit configured to cause a display unit to display the state data as a time series graph based on the generation time information,
wherein when at least some of the series of frame images are to be played back as a moving image, the display control unit causes an index which represents a position corresponding to the displayed frame image to be displayed on the graph.

12. The apparatus according to claim 11, wherein the state data is one of data of a spirometer, data of a cardiograph, and data of a capnography device.

13. An image processing method comprising:
extracting at least one region of interest with respect to each frame image of a series of frame images of an object;
calculating a feature amount of the region of interest; and
controlling to cause a display unit to display the feature amount as a time series graph,
wherein when at least some of the series of frame images are to be played back as a moving image, an index which represents a position corresponding to the displayed frame image is displayed on the graph in the controlling.

14. An image processing method comprising:
obtaining a series of frame images of an object;
obtaining state data which indicates a state of the object and generation time information of the state data; and
controlling to cause a display unit to display the state data as a time series graph based on the generation time information,
wherein when at least some of the series of frame images are to be played back as a moving image, an index which represents a position corresponding to the displayed frame image is displayed on the graph in the controlling.

15. A non-transitory computer-readable storage medium storing a program to cause a computer to execute an image processing method comprising:
extracting at least one region of interest with respect to each frame image of a series of frame images of an object;
calculating a feature amount of the region of interest; and
controlling to cause a display unit to display the feature amount as a time series graph,
wherein when at least some of the series of frame images are to be played back as a moving image, an index which represents a position corresponding to the displayed frame image is displayed on the graph in the controlling.

16. A non-transitory computer-readable storage medium storing a program to cause a computer to execute an image processing method comprising:
obtaining a series of frame images of an object;
obtaining state data which indicates a state of the object and generation time information of the state data; and
controlling to cause a display unit to display the state data as a time series graph based on the generation time information,
wherein when at least some of the series of frame images are to be played back as a moving image, an index which represents a position corresponding to the displayed frame image is displayed on the graph in the in the controlling.
